(19) United States
(12) Patent Application Publication
(10) Pub. No.: US 2024/0138797 A1
(43) Pub. Date: May 2, 2024

(54) INFORMATION PROCESSING APPARATUS AND CONTROL METHOD THEREOF, RADIATION IMAGING SYSTEM, AND STORAGE MEDIUM

(71) Applicant: CANON KABUSHIKI KAISHA, Tokyo (JP)

(72) Inventors: Koki FUJIMORI, Kanagawa (JP); Hiroyuki OMI, Kanagawa (JP)

(21) Appl. No.: 18/475,273

(22) Filed: Sep. 27, 2023

(30) Foreign Application Priority Data
Oct. 26, 2022 (JP) 2022-171582

(51) Int. Cl. A61B 6/00; G06T 7/00; G06T 7/33

(52) U.S. Cl. CPC A61B 6/5241; A61B 6/482; A61B 6/505; A61B 6/54; G06T 7/0012; G06T 7/33; G06T 2207/10116; G06T 2207/20081; G06T 2207/20224

(57) ABSTRACT

An information processing apparatus determines, for each of a first radiation image corresponding to a first radiation energy and a second radiation image corresponding to a second radiation energy different from the first radiation energy, a first bone area indicating a predetermined bone portion, performs an alignment between a first partial area including the first bone area in the first radiation image and a second partial area including the first bone area in the second radiation image, and obtains a bone mineral density of the first bone area based on a difference image including the first bone area, the difference image is obtained based on a result of the alignment.

### BACKGROUND OF THE INVENTION

### Field of the Invention

The present invention relates to an information processing apparatus and a control method thereof, a radiation imaging system, and a storage medium.

### Description of the Related Art

In a dual energy X-ray absorptiometry (DXA) method, which is a common method of measuring a bone mineral density, X-rays of two different energies (radiation qualities) are obtained, and a bone mineral density is measured according to a difference in absorption rate between bone and soft tissue. Bones for which a bone mineral density is measured by the DXA method are, for example, a lumbar spine and a femur. The DXA method is advantageous in that accurate and fast measurement is possible for both body parts and a radiation dosage is small.

Here, as an X-ray imaging method in which X-rays of two different energies are obtained, there is a two-shot method. In this imaging method, X-ray imaging is carried out two times with a subject being irradiated with X-rays of different energies. Therefore, in the two-shot method, there is a problem that a time difference between the shots cannot be avoided, and artifacts occur due to movements of the subject in that time difference. It is possible to obtain an image in which artifacts are reduced by accurately aligning the X-ray images obtained by the two instances of shooting. Japanese Patent Laid-Open No. 2017-63936 discloses, as an example of aligning two images, a method of aligning images by setting and associating characteristic points (landmarks) on bones extracted from respective images. In addition, Japanese Patent Laid-Open No. 2009-273638 discloses a method of aligning the entire images by performing template matching for each of grid points set at predetermined intervals across the entire images.

Conventional image alignment is performed across the entire images. When entire images are aligned, there cases where accuracy of the overall alignment improves but accuracy of alignment of areas that are necessary for bone mineral density measurement is insufficient.

### SUMMARY OF THE INVENTION

According to one aspect of the present invention, there is provided an information processing apparatus comprising: a determination unit configured to determine, for each of a first radiation image corresponding to a first radiation energy and a second radiation image corresponding to a second radiation energy different from the first radiation energy, a first bone area indicating a predetermined bone portion; an alignment unit configured to perform an alignment between a first partial area including the first bone area in the first radiation image and a second partial area including the first bone area in the second radiation image; and an obtaining unit configured to obtain a bone mineral density of the first bone area based on a difference image including the first bone area, the difference image being obtained based on a result of the alignment.

Further features of the present invention will become apparent from the following description of exemplary embodiments with reference to the attached drawings.

### BRIEF DESCRIPTION OF THE DRAWINGS

FIG. 1 is a block diagram illustrating an example of a functional configuration of a bone mineral density measurement apparatus according to a first embodiment.

FIG. 2 is a diagram illustrating an example of a hardware configuration of a radiation imaging system according to the first embodiment.

FIG. 3 is a flowchart for explaining bone mineral density measurement processing according to the first embodiment.

FIG. 4 is a diagram illustrating an example of noise removal by a median filter.

FIGS. 5A and 5B are diagrams illustrating learning model machine learning and bone mineral density measurement area setting processing.

FIG. 6 is a diagram illustrating template image cutting according to the first embodiment.

FIG. 7 is a diagram illustrating an example of search area setting according to the first embodiment.

FIG. 8 is a diagram for explaining template matching according to the first embodiment.

FIG. 9 is a diagram illustrating a bone image generation procedure according to the first embodiment.

FIG. 10 is a diagram illustrating a bone mineral density measurement area obtainment method according to a second embodiment.

FIG. 11 is a diagram illustrating machine learning of a learning model for extracting a bone mineral density measurement area constituting of a second lumbar vertebra to a fourth lumbar vertebra.

FIG. 12 is a diagram illustrating an example of a template and search area setting according to the second embodiment.

FIGS. 13A and 13B are diagrams illustrating two-stage alignment processing according to a third embodiment.

FIG. 14 is a flowchart for explaining bone mineral density measurement processing according to a fourth embodiment.

FIG. 15 is a diagram illustrating an example of display of a warning message according to the fourth embodiment.

FIG. 16 is a diagram for explaining bone mineral density calculation processing for when the lumbar spine and the femora are simultaneously set as a bone mineral density measurement area according to a fifth embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

According to the following embodiments, a bone mineral density measurement apparatus for improving alignment accuracy for an area necessary for bone mineral density measurement is disclosed.

### First Embodiment

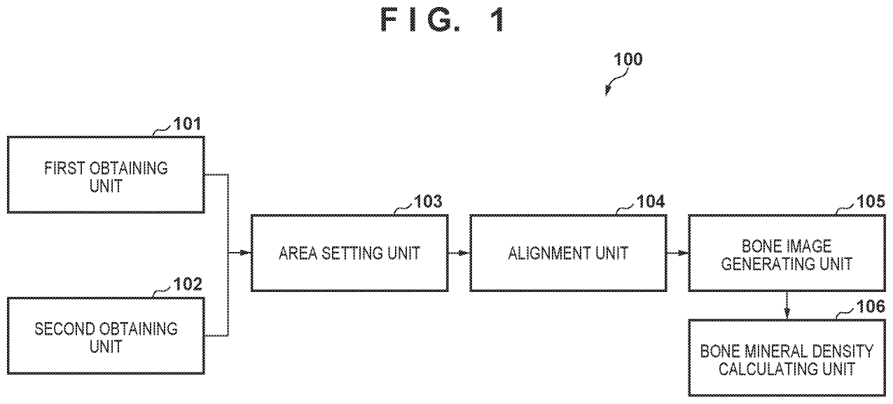

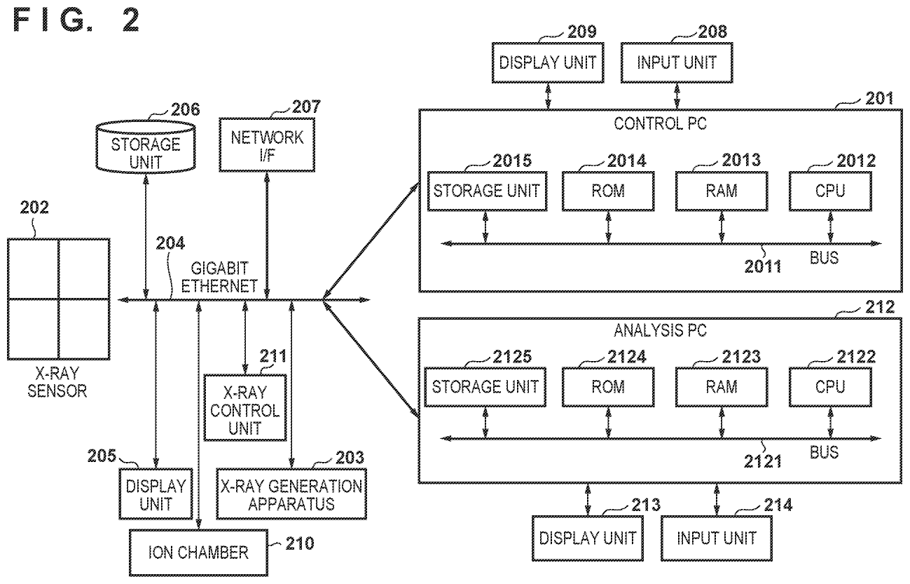

An example of a functional configuration of a bone mineral density measurement apparatus according to a first embodiment is illustrated in FIG. 1. A bone mineral density measurement apparatus 100 includes a first obtaining unit 101, a second obtaining unit 102, an area setting unit 103, an alignment unit 104, a bone image generating unit 105, and a bone mineral density calculating unit 106. The first obtaining unit 101 obtains a first radiation image, which corresponds to a first radiation energy, and the second obtaining unit 102 obtains a second radiation image, which corresponds to a second radiation energy different from the first radiation energy. Specifically, in the present embodiment, X-rays are used as radiation. Accordingly, the first obtaining unit 101 takes, as input, a first X-ray image obtained from an X-ray sensor 202 (FIG. 2) and outputs a preprocessed first X-ray image. The second obtaining unit 102 takes, as input, a second X-ray image obtained from the X-ray sensor 202 and outputs a preprocessed second X-ray image. The first X-ray image corresponds to a first X-ray energy, and the second X-ray image corresponds to a second X-ray energy different from the first X-ray energy.

The area setting unit 103 extracts a bone area for each of the first X-ray image and the second X-ray image and sets the extracted bone areas as bone mineral density measurement areas. The alignment unit 104 obtains a shift amount (dxmin, dymin) between the bone mineral density measurement area of the first X-ray image and the bone mineral density measurement area of the second X-ray image. In the alignment, partial areas determined based on the respective bone mineral density measurement areas set by the area setting unit 103 for the first X-ray image and the second X-ray image are used. The bone image generating unit 105 generates a difference image, which is a bone image which includes the aforementioned extracted bone areas, from the first X-ray image and the second X-ray image aligned according to the shift amount obtained by the alignment unit 104. The bone mineral density calculating unit 106 obtains a bone mineral density of the aforementioned extracted bone area based on the bone image (difference image) generated by the bone image generating unit 105.

FIG. 2 is a block diagram illustrating an example of a configuration of a radiation imaging system according to the first embodiment. In this example, an X-ray imaging system is indicated as the radiation imaging system. In the X-ray imaging system of the present embodiment, for example, an analysis PC 212, which is an example of an information processing apparatus, functions as the bone mineral density measurement apparatus 100. A control PC 201, the X-ray sensor 202, an X-ray generation apparatus 203, a display unit 205, a storage unit 206, a network interface unit 207, an ion chamber 210, an X-ray control unit 211, and the analysis PC 212 are connected by Gigabit Ethernet 204. The X-ray sensor 202 is an example of a radiation imaging apparatus for performing radiation imaging In addition, the X-ray generation apparatus 203 is an example of a radiation generation apparatus for generating radiation of different radiation energies according to a setting of a tube voltage or the like by the X-ray control unit 211. The Gigabit Ethernet 204 is an example of a signal transmission medium; the signal transmission medium is not limited to this and may be, for example, a controller area network (CAN), optical fiber, or the like.

The control PC 201 includes a central processing unit (CPU) 2012, a random access memory (RAM) 2013, a read-only memory (ROM) 2014, and a storage unit 2015. In addition, these components are connected to a bus 2011. In addition, an input unit 208 is connected to the control PC 201 via an interface, such as USB or PS/2, and a display unit 209 is connected to the control PC 201 via an interface, such as DisplayPort or DVI. The control PC 201 transmits commands to the X-ray sensor 202, the display unit 205, and the like. In the control PC 201, processing contents for each imaging mode are stored in the storage unit 2015 as software modules and are read into the RAM 2013 as necessary and then executed by the CPU 2012. X-ray images obtained by the X-ray sensor 202 and processed X-ray images are stored in the storage unit 2015 of the control PC 201 or the storage unit 206 which is external to the control PC 201. For example, the control PC 201 controls X-ray imaging by using the X-ray control unit 211. More specifically, the control PC 201 obtains an X-ray image that corresponds to the first X-ray energy by performing imaging in which the X-ray sensor 202 is used while causing the X-ray generation apparatus 203 to generate X-rays according to the first X-ray energy. In addition, the control PC 201 obtains an X-ray image that corresponds to the second X-ray energy different from the first X-ray energy by performing imaging in which the X-ray sensor 202 is used while causing the X-ray generation apparatus 203 to generate X-rays according to the second X-ray energy. The obtained X-ray images are stored in the storage unit 206 and are read out and analyzed by the analysis PC 212.

The analysis PC 212 includes, for example, a CPU 2122, a RAM 2123, a ROM 2124, and a storage unit 2125. In addition, these components are connected to a bus 2121. An input unit 213 is connected to the analysis PC 212 via an interface, such as USB or PS/2, and a display unit 214 is connected to the analysis PC 212 via DisplayPort or DVI. In the analysis PC 212, bone mineral density calculation processing contents and bone mineral density analysis report creation contents are stored in the storage unit 2125 as software modules and are read into the RAM 2123 as necessary and then executed by the CPU 2122. The processed X-ray images and the report (obtained bone mineral density) are transmitted to and stored in the external storage unit 206.

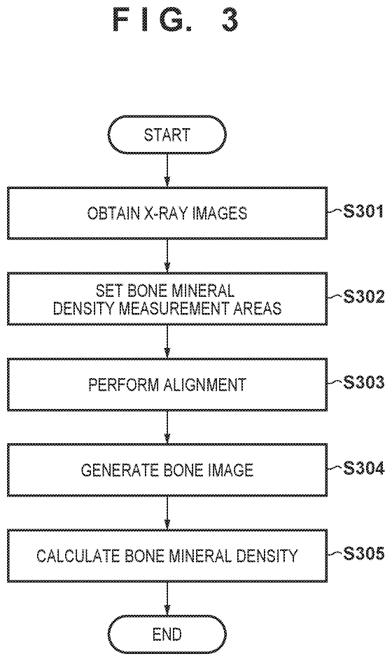

The analysis PC 212 is an example of an information processing apparatus that functions as the bone mineral density measurement apparatus 100, and each functional unit block illustrated in FIG. 1 can be realized by the CPU 2122 executing a software module stored in the storage unit 2125. Of course, at least some of the functional blocks illustrated in FIG. 1 may be realized by dedicated hardware, such as an image processing board, or may be realized by the CPU 2122 (software) and hardware cooperating. Alternatively, some of the functions of the bone mineral density measurement apparatus 100 may be realized by the control PC 201. Details of the operation of the bone mineral density measurement apparatus of the present embodiment in which the above components are included will be described below. In the following embodiments, processing for calculating a bone mineral density of a lumbar spine will be described. FIG. 3 is a flowchart for explaining bone mineral density measurement processing according to the first embodiment.

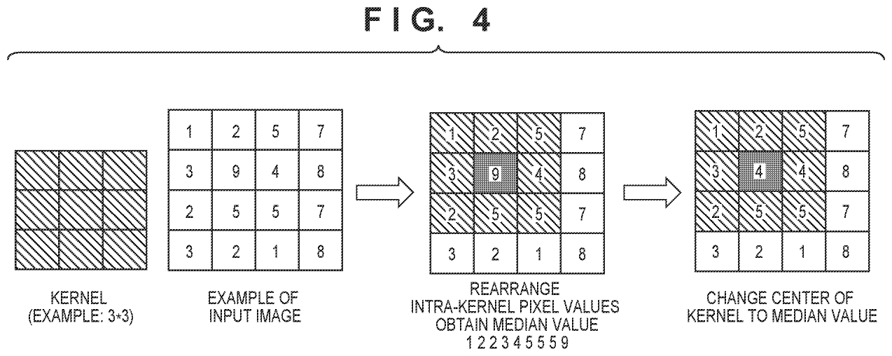

In step S301, the first obtaining unit 101 obtains a first lumbar spine image by preprocessing the first X-ray image obtained by capturing an image of a lumbar spine portion of a subject with the first X-ray energy. Here, the preprocessing refers to processing for correcting characteristics of the X-ray sensor 202 and includes offset correction (dark current correction), gain correction, defect correction, and the like. In addition, the pre-processing may include noise removal. As a method of noise removal, a method in which a median filter is used can be given as an example. FIG. 4 is a diagram illustrating noise removal by a median filter as an example of such noise removal. In noise removal by a median filter, a kernel of a desired size (3×3 in FIG. 4) is used, a median value of intra-kernel-area pixel values of an input image is calculated by rearranging the pixel values in ascending order, and a center pixel value of the kernel is replaced with the calculated median value. By repeatedly performing this processing across the entire image, it is possible to eliminate pixel values, such as noise, that are significantly different compared to those of surrounding pixels. The second obtaining unit 102 obtains a second lumbar spine image by performing pre-processing similar to that of the first obtaining unit 101 on the second X-ray image obtained by capturing an image of the lumbar spine portion of the subject with the second X-ray energy (radiation quality) different from the first X-ray energy.

In step S302, the area setting unit 103 sets a bone mineral density measurement area for each of the first lumbar spine image and the second lumbar spine image. The area setting unit 103 determines a bone area (lumbar spine area) of a lumbar spine portion for each of the first lumbar spine image and the second lumbar spine image and then generates a first measurement area image and a second measurement area image. Examples of a method of extracting a lumbar spine area include using a learning model generated by supervised machine learning. Specifically, a learning model is generated using a convolutional neural network (hereinafter, CNN).

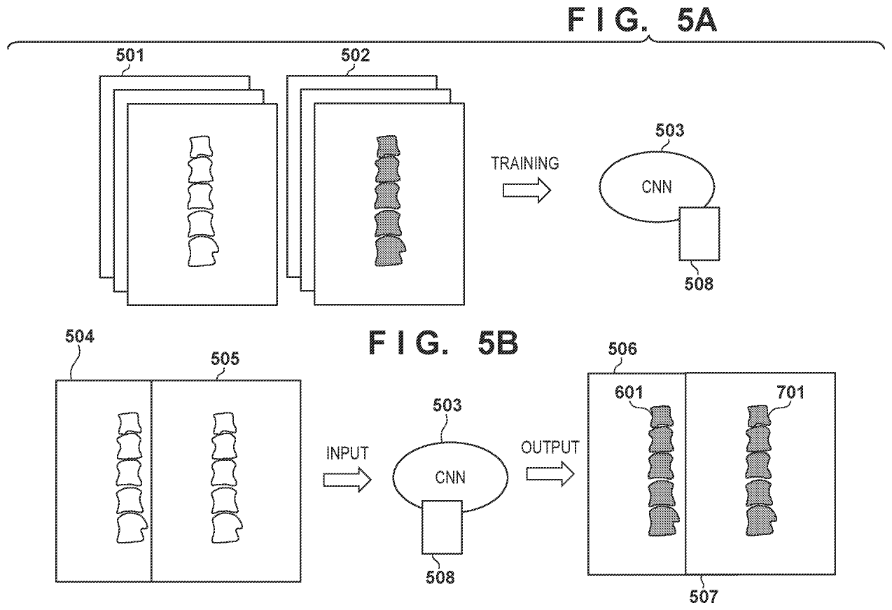

FIGS. 5A and 5B are diagrams for explaining training and inference of a function for extracting a lumbar spine area in the area setting unit 103. In FIGS. 5A and 5B, to eliminate complexity in the illustration, only a bone area of the lumbar spine is illustrated in lumbar spine images and measurement area images. In actuality, other bone portions, such as a thoracic spine and a sacrum, and soft tissue are captured in each image, in addition to the lumbar spine. This is similar for other drawings.

FIG. 5A illustrates a training-time operation of the function for extracting a lumbar spine area. Learned data 508 is generated by a learning model 503 (CNN) learning pre-processed lumbar spine images 501 and correct images 502 in which a lumbar spine area (bone area used for bone mineral density measurement) has been manually labeled in the lumbar spine images 501. Hereinafter, the learning model 503 to which the learned data 508 is applied is referred to as a trained model. The lumbar spine images 501 used for training the learning model 503 include both images that correspond to the first X-ray energy and images that correspond to the second X-ray energy different from the first X-ray energy. Training in which both lumbar spine images that correspond to the first X-ray energy and lumbar spine images that correspond to the second X-ray energy are used improves robustness against different tube voltages at the time of inference. In addition, at the time of inference, the same trained model can be used for a lumbar spine image that corresponds to the first X-ray energy and for a lumbar spine image that corresponds to the second X-ray energy. One trained model thus being sufficient is advantageous for simplifying the system and reducing memory.

FIG. 5B illustrates an inference-time operation for a lumbar spine area in which a trained model is used. The area setting unit 103 inputs a first lumbar spine image 504, which is a pre-processed first X-ray image, and a second lumbar spine image 505, which is a pre-processed second X-ray image, into a trained model. With this, a lumbar spine area, which is a bone mineral density measurement area, is inferred for each lumbar spine image and then a first measurement area image 506 and a second measurement area image 507 are generated. More specifically, the first measurement area image 506 is generated by applying, to the first lumbar spine image 504, information (position, size) of a bone mineral density measurement area 601 inferred for the first lumbar spine image 504. Similarly, the second measurement area image 507 is generated by applying, to the second lumbar spine image 505, information (position, size) of a bone mineral density measurement area 701 inferred for the second lumbar spine image 505.

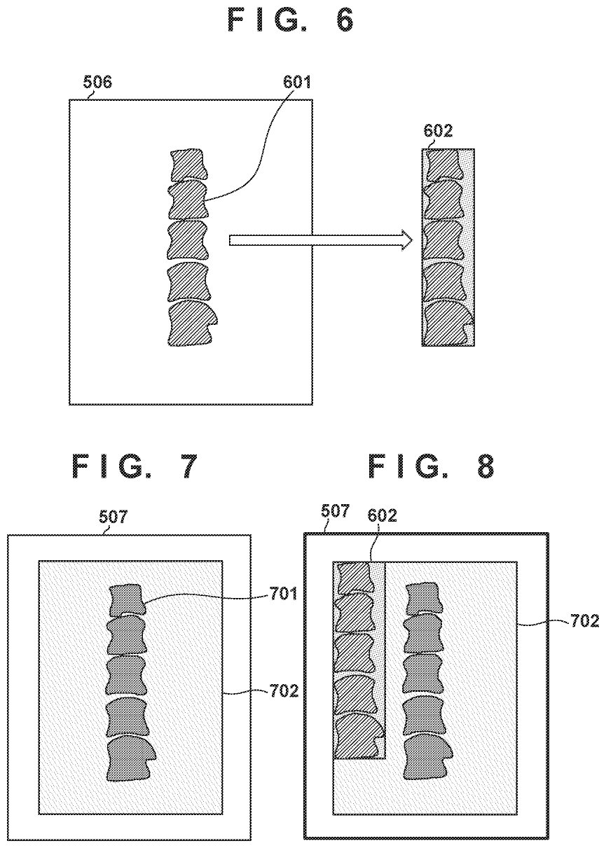

Returning to FIG. 3, the alignment unit 104 performs alignment processing by using the first measurement area image 506 and the second measurement area image 507 (step S303). In the alignment processing of the present embodiment, for example, template matching is used, and a shift amount (dxmin, dymin) (translation amount) for alignment is obtained for two images to be aligned. The shift amount obtained here is a moving amount for matching the bone mineral density measurement area of the first measurement area image 506 and the bone mineral density measurement area of the second measurement area image 507. A template used for template matching is generated from the first measurement area image 506, and a search area is generated from the second measurement area image 507. Examples of a method of creating a template include, as illustrated in FIG. 6, cutting out, as a template 602, a partial area defined by a rectangle in which the bone mineral density measurement area 601 set in the first measurement area image 506 is circumscribed.

In addition, a search area is set based on the bone mineral density measurement area set in the second measurement area image 507. For example, as illustrated in FIG. 7, in the second measurement area image 507 in which the bone mineral density measurement area 701 has been set, a partial area that includes the bone mineral density measurement area 701 and is larger than the template 602 is set as a search area 702. Regarding the size of the search area 702, for example, a size suitable for performing template matching in which the template 602 is used is set based on the size of the bone mineral density measurement area 701. Alternatively, the size of the search area 702 may be set based on the size of the template 602. In such a case, for example, the size of the search area 702 is obtained by increasing the vertical and horizontal sizes of the template 602 at a predetermined proportion. At this time, calculation cost is suppressed by making the search area 702 as small as possible. The position of the search area 702 may be determined such that, for example, the center of gravity of the bone mineral density measurement area 701 and the center of gravity of the search area 702 match.

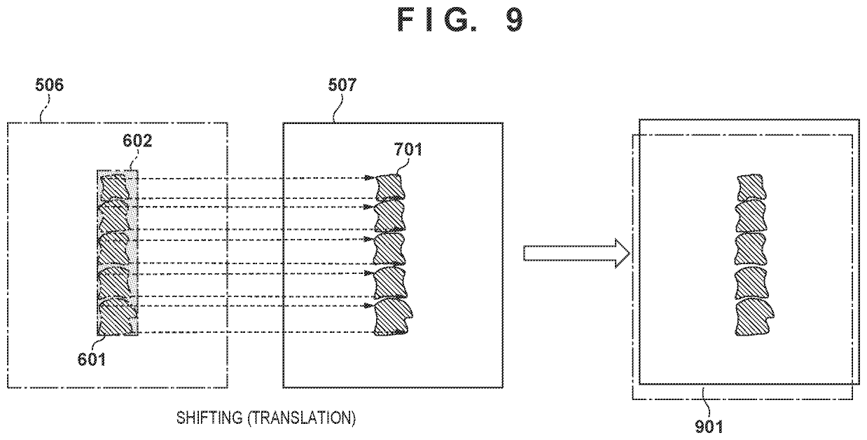

Then, the alignment unit 104 performs template matching by using the template 602 and the search area 702 obtained as described above. At this time, as illustrated in FIG. 8, a similarity is evaluated at each position of the template while shifting the template 602 in the search area 702 of the second measurement area image 507. Examples of a method of evaluating a similarity include a sum of squared differences (SSD) in which a similarity is evaluated by a “sum of absolute values of differences between pixel values”. For example, when it is assumed that a pixel value of the second measurement area image 507 is I(x, y), a pixel value of the template 602 is T(x, y), the width of the template 602 is w, and the height of the template 602 is h, an SSD value at a scan position of dx, dy can be calculated using Equation (1). The smaller this SSD value, the higher the similarity that it represents. The alignment unit 104 outputs an amount of shift (dxmin, dymin) between the first measurement area image 506 and the second measurement area image 507 at the position of the template 602 at which the smallest SSD value is obtained.

$$\begin{matrix}[ EQUATION 1 ] & \\ S S D ( {d}_{x} , {d}_{y} ) = {\sum }_{x = 0}^{w - 1} {\sum }_{y = 0}^{h - 1} {( I ( {d}_{x} + x , {d}_{y} + y ) - T ( x , y ) )}^{2} & ( 1 )\end{matrix}$$

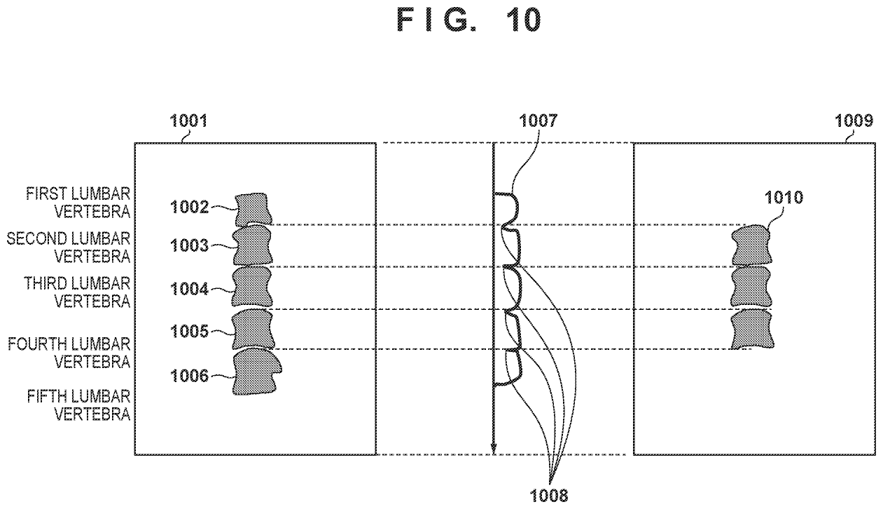

The bone image generating unit 105 generates a bone image by using a result of the alignment by the alignment unit 104 (step S304). Here, the alignment result is a parameter for aligning the bone mineral density measurement area of the first measurement area image 506 and the bone mineral density measurement area of the second measurement area image 507 and is, for example, the above-described shift amount (dxmin, dymin). For example, as illustrated in FIG. 9, the bone image generating unit 105 aligns both images by shifting the first measurement area image 506 with respect to the second measurement area image 507 based on the shift amount (dxmin, dymin) obtained by the alignment unit 104. The bone image generating unit 105 obtains a difference image (bone image) by taking a difference between the aligned first measurement area image 506 and second measurement area image 507. In the present embodiment, the bone image generating unit 105 generates a difference image (bone image 901) by obtaining a logarithmic difference for the aligned first measurement area image 506 and second measurement area image 507. For example, the aligned second lumbar spine image is set to be a high energy image logy_{high}, which corresponds to a high X-ray energy, and the aligned first lumbar spine image is a low energy image logy_{low}, which corresponds to a low X-ray energy. Then, a bone image Δlog, which is a difference image thereof, is as in Equation (2).

[EQUATION 2]

Δlogy=log y_{high}−log y_{low} (2)

The bone mineral density calculating unit 106 calculates a bone mineral density BMD by using the bone image 901 generated by the bone image generating unit 105 (step S305). In the calculation of the bone mineral density BMD, the bone mineral density calculating unit 106 calculates, as an average value X_{bone} of a bone area, an average value of pixel values of a portion that corresponds to the bone mineral density measurement area 601 (or the bone mineral density measurement area 701) in the bone image 901. Next, the bone mineral density calculating unit 106 calculates, as an average value Y_{softtissue} of a background area, an average value of a soft tissue area in the bone image 901. The average value of the soft tissue area is obtained, for example, by setting a rectangular area at a location (location in which bones are not included) away from the lumbar spine by a specific distance and then calculating an average value of pixel values in that rectangular area. Then, the bone mineral density BMD is calculated by taking a difference by subtracting the average value Y_{softtissue} of the background area from the average value X_{bone} of the bone area as in Equation (3).

[EQUATION 3]

BMD=X̅_{bone}Y̅_{softtissue} (3)

In the above-described embodiment, only shifting (translation) is used as linear alignment by the alignment unit 104; however, the present invention is not limited to this. Other linear alignment, such as rotation or scaling of images, or a combination thereof may be used. In addition, in the above-described embodiment, the bone image 901 is obtained by aligning the first measurement area image 506 and the second measurement area image 507; however, the present invention is not limited to this. It is obvious that the bone image 901 may also be obtained by aligning the first lumbar spine image 504 and the second lumbar spine image 505 based on the above-described shift amount. In addition, in the present embodiment, processing for calculating a bone mineral density in the lumbar spine has been described; however, the present invention is not limited to this and can be applied to, for example, processing for calculating a bone mineral density in a femur. In such a case, the above-described bone mineral density measurement area is simply changed to that of a femur, and other than that is similar.

As described above, according to the first embodiment, a template and a search area are set based on respective bone mineral density measurement areas determined for two X-ray images (for example, the first and second lumbar spine images). Thus, a partial area to be used for alignment is limited based on the determined bone mineral density measurement areas, and accurate alignment for bone mineral density measurement area can be realized. As a result, the accuracy of bone mineral density measurement is improved. cl Second Embodiment

In the first embodiment, an example in which a bone mineral density is calculated using, as a bone mineral density measurement area, a bone area (e.g., the entire lumbar spine) determined by a learning model has been described. In a second embodiment, a configuration in which a bone mineral density is calculated using, as a bone mineral density measurement area, a portion of the bone area determined by the learning model, for example, will be described. A functional configuration, a hardware configuration, and the overall bone mineral density measurement processing of a bone mineral density measurement apparatus of the second embodiment are similar to those of the first embodiment (FIGS. 1, 2, and 3). Hereinafter, bone mineral density measurement processing according to the second embodiment will be described with reference to FIG. 3 again.

In step S301, the first obtaining unit 101 obtains a first lumbar spine image, which is a first X-ray image, and then obtains a preprocessed first lumbar spine image by preprocessing the obtained lumbar spine image. In addition, the second obtaining unit 102 obtains a second lumbar spine image, which is a second X-ray image, and then obtains a preprocessed second lumbar spine image by preprocessing the obtained lumbar spine image.

The area setting unit 103 sets a bone mineral density measurement area in each of the first lumbar spine image and the second lumbar spine image (step S302). A bone mineral density at the lumbar spine may be measured using a portion (a group of lumbar vertebrae selected from among five lumbar vertebrae) of rather than the entire lumbar spine. For example, there are cases, such as a case in which a bone mineral density is first measured at a second lumbar vertebra, a third lumbar vertebra, and a fourth lumbar vertebra and then a bone mineral density is measured after adding a first lumbar vertebra. A user can set, as a measurement range, which vertebra of the first to fifth lumbar vertebrae is to be used for bone mineral density measurement. In the present embodiment, a bone mineral density measurement area is set according to the measurement range set by the user.

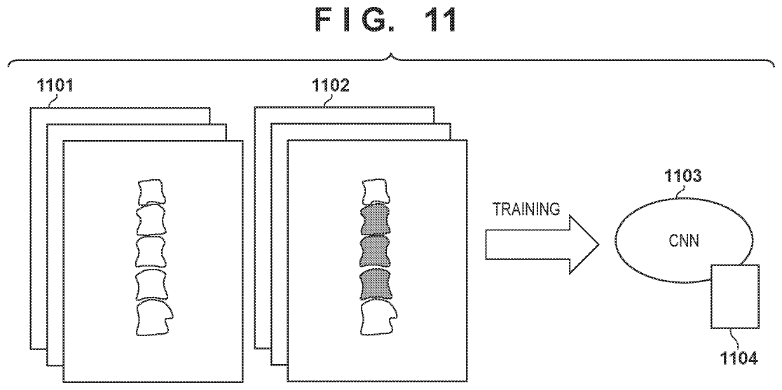

FIG. 10 is a diagram illustrating bone mineral density measurement area setting processing by the area setting unit 103 according to the second embodiment. Hereinafter, when collectively referring to the first lumbar spine image and the second lumbar spine image, they are simply referred to as lumbar spine images. Similarly, when collectively referring to the first measurement area image and the second measurement area image, they are simply referred to as measurement area images. A measurement area image 1001 is an image obtained by extracting a bone area of the entire lumbar spine and is obtained by inputting a pre-processed lumbar spine image into a trained model, similarly to the first embodiment (FIGS. 5A and 5B). The area setting unit 103 separates the extracted bone area of the entire lumbar spine for each vertebral body. The lumbar spine consists of five vertebral bodies and so, as illustrated in FIG. 10, the lumbar spine is separated into five vertebral bodies, and from the top, a first lumbar vertebra 1002, a second lumbar vertebra 1003, a third lumbar vertebra 1004, a fourth lumbar vertebra 1005, and a fifth lumbar vertebra 1006 are set in that order. As a method of separating the lumbar spine into individual vertebral bodies, a bone area for each vertebral body is determined based on a profile obtained by adding, in a predetermined direction, pixel values of the bone area (the entire lumbar spine) set in the measurement area image 1001. For example, as illustrated in FIG. 10, the area setting unit 103 creates a profile 1007 in which pixel values of a bone area of the lumbar spine have been added in a direction (lateral direction in the example of FIG. 10) substantially vertical to the arrangement of the lumbar spine in the measurement area image 1001. Then, the area setting unit 103 separates the vertebral bodies by extracting positions 1008, which are local minimum values in the profile 1007, as gaps. The lumbar vertebrae to be used as a bone mineral density measurement area are extracted based on the separation result thus obtained. In the present embodiment, the second lumbar vertebra 1003, the third lumbar vertebra 1004, and the fourth lumbar vertebra 1005 are set by the user as a bone mineral density measurement range (bone mineral density measurement area). That is, an area constituting of the second to fourth vertebrae among five separated vertebral bodies is set as a bone mineral density measurement area 1010. A measurement area image 1009 in which the bone mineral density measurement area 1010 is set is thus obtained. The bone mineral density measurement range is set by the user designating a desired combination from among five vertebrae constituting the lumbar spine.

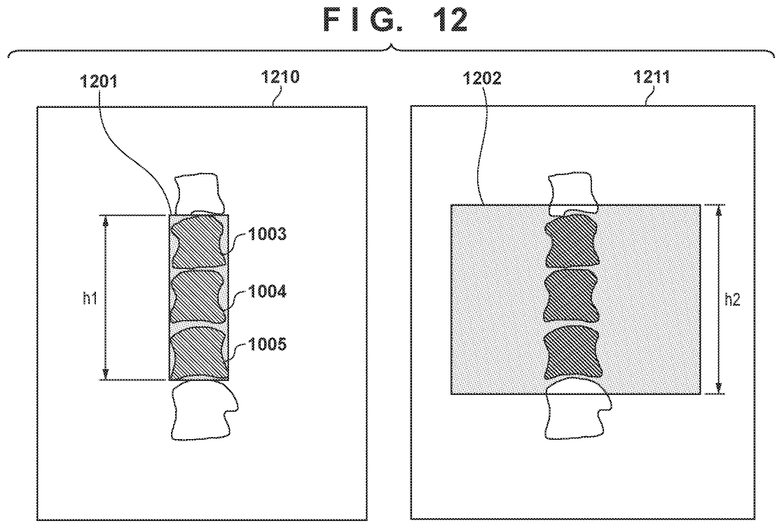

In addition, a measurement area image in which a portion of the lumbar spine set as a measurement range is set as a bone mineral density measurement area may be obtained from the first lumbar spine image and the second lumbar spine image by using machine learning similar to that of the first embodiment. In such a case, a flow of processing is similar to that of the first embodiment. However, in such a case, it is necessary to prepare a trained model for each combination of vertebrae that corresponds to a measurement range, and correct images to be used for training need to be changed according to the combination of vertebral bodies. For example, when the second lumbar vertebra to fourth lumbar vertebra are used, as illustrated in FIG. 11, training is performed using lumbar spine images 1101 and correct images 1102 in which the second lumbar vertebra 1003, the third lumbar vertebra 1004, and the fourth lumbar vertebra 1005 have been labeled.

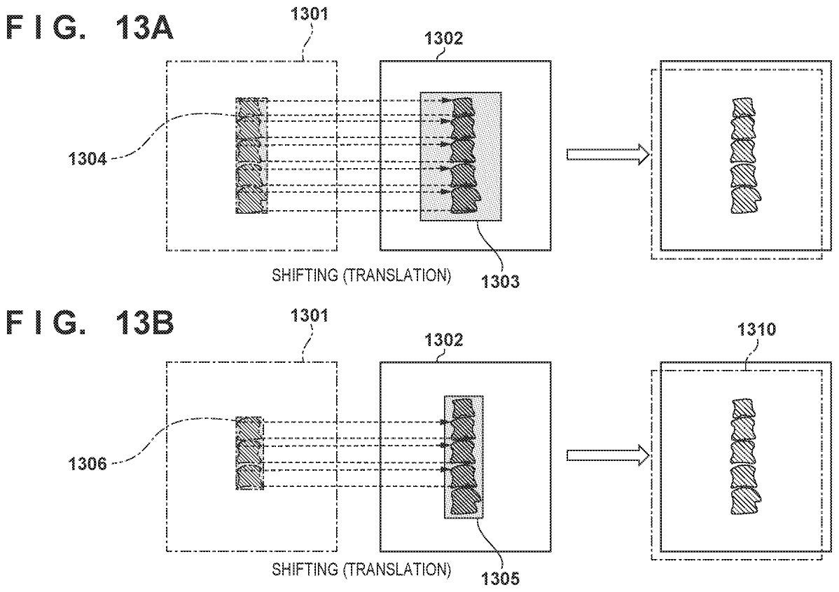

Next, the alignment unit 104 performs alignment processing by using the first measurement area image and the second measurement area image (step S303). In the present embodiment, the alignment is performed based on a bone mineral density measurement area corresponding to a set measurement range. FIG. 12 is a diagram for explaining a template and a search area setting used for template matching according to the second embodiment. A case where the second lumbar vertebra, the third lumbar vertebra, and the fourth lumbar vertebra are set as a measurement range as described above will be described. In the above-described step S302, a first measurement area image 1210 in which a bone area of the second lumbar vertebra, the third lumbar vertebra, and the fourth lumbar vertebra are set as a bone mineral density measurement area in the first lumbar spine image 504 is generated. The alignment unit 104 cuts out a circumscribing rectangle that surrounds the bone mineral density measurement area (the second lumbar vertebra, the third lumbar vertebra, and the fourth lumbar vertebra) from the first measurement area image 1210 as a template 1201. In addition, a search area 1202 is set in a second measurement area image 1211 in which the bone area of the second lumbar vertebra, the third lumbar vertebra, and the fourth lumbar vertebra are set as the bone mineral density measurement area in the second lumbar spine image 505. In order to prevent matching with a vertebra other than the vertebrae in the measurement range, the search area 1202 is set to be small in a direction (vertical direction in FIG. 12) parallel to the arrangement of the lumbar spine so as to reduce a shift amount of the template 1201 in the direction parallel to the arrangement the lumbar spine. For example, a search area in the direction parallel to the arrangement of the lumbar spine is set to be a sum of a vertical size of the template and a half of a vertical size of the template divided by the number of vertebral bodies included in the template. In the example of FIG. 12, the number of vertebral bodies included in the template 1201 is three and the vertical size is h1, and so, a vertical size h2 of the search area is h2=h1+(h1/3/2). The alignment unit 104 performs the alignment similarly to the first embodiment by using the template 1201 and the search area 1202 and then obtains a shift amount (dxmin, dymin). A horizontal size of the search area 1202 may be set similarly to the first embodiment.

Then, the bone image generating unit 105 generates a bone image by using the first measurement area image 1210, the second measurement area image 1211, and the above-described shift amount (step S304), and the bone mineral density calculating unit 106 calculates a bone mineral density of the bone mineral density measurement area by using the bone image. Steps S304 and S305 are similar to those of the first embodiment.

As described above, in the second embodiment, alignment is performed not on the entire lumbar spine area as in the first embodiment but by further limiting a bone mineral density measurement area, and so, the accuracy is further improved. In addition, the user can set, as a bone density measurement area, a desired combination of vertebrae among lumbar vertebrae and then obtain a bone mineral density.

In the second embodiment, an example in which lumbar vertebrae are used as a bone mineral density measurement area has been described; however, the present invention is not limited to this, and for example, a femur may be used as a bone mineral density measurement area. In a case of a femur, the accuracy is increased by limiting the measurement range (bone mineral density measurement area) to a head portion and a trochanter portion and then performing alignment. In the above-described example, a bone mineral density measurement area is set according to a system-set measurement range; however, a configuration may be taken such that when the user further corrects the measurement range thereafter, bone mineral density measurement is repeated using the corrected measurement range as the bone mineral density measurement area.

### Third Embodiment

In the first embodiment and the second embodiment, template matching according to a template and a search area set based on a bone mineral density measurement area has been described as an alignment method. In a third embodiment, two-stage template matching is performed: global matching based on a bone area larger than a bone mineral density measurement area and local matching based on the bone mineral density measurement area. A functional configuration, a hardware configuration, and the overall bone mineral density measurement processing of a bone mineral density measurement apparatus of the third embodiment are similar to those of the first embodiment (FIGS. 1, 2, and 3). Hereinafter, bone mineral density measurement processing according to the third embodiment will be described with reference to FIG. 3 again.

Step S301 to step S302 are similar to those of the second embodiment. Next, the alignment unit 104 performs alignment processing by using the first measurement area image and the second measurement area image (step S303). In the alignment processing of the third embodiment, global matching in which alignment is performed based on a bone area of the entire lumbar spine is first performed. Then, after global matching, local matching in which alignment is performed in a bone area (bone mineral density measurement area) that corresponds to a measurement range set by the user or set in advance by the system is performed. In the third embodiment, a two-stage alignment of global matching and local matching is thus performed.

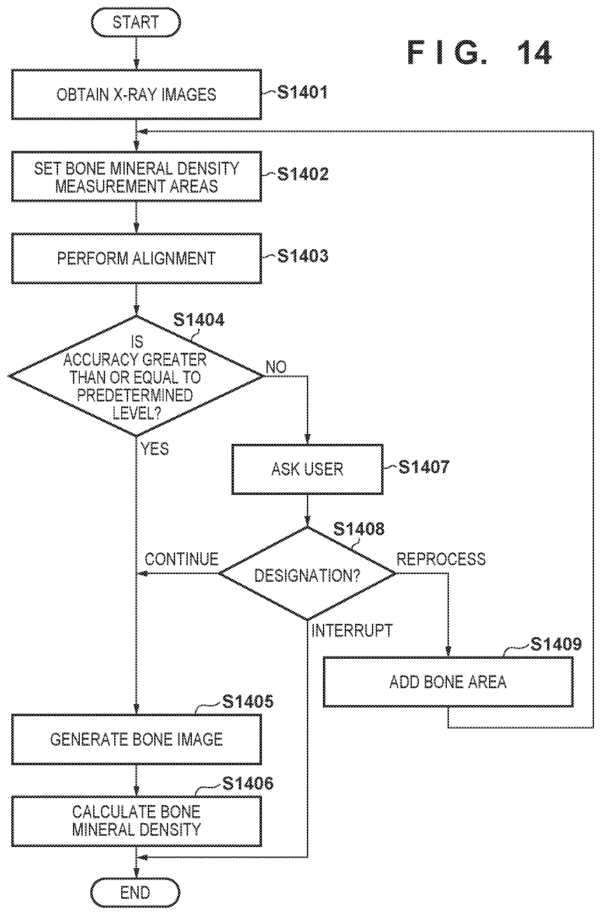

The two-stage alignment according to the third embodiment will be described with reference to FIGS. 13A and 13B. First, as illustrated in FIG. 13A, the alignment unit 104 performs global matching by using the entire extracted lumbar spine. Similarly to the alignment processing of the first embodiment, the alignment unit 104 performs alignment processing by shifting, in a search area 1303 of a second measurement area image 1302, a template 1304 that includes the entire lumbar spine obtained from a first measurement area image 1301. A global shift amount (dxminG, dyminG) is thus calculated. The template 1304 of the entire lumbar spine is a template that includes all of the first lumbar vertebra to the fifth lumbar vertebra used in the first embodiment and corresponds to the template 602 of FIG. 6. Similarly, the search area 1303 corresponds to the search area 702 of FIG. 7.

Next, local matching illustrated in FIG. 13B is performed. A template 1306 for local matching is a template that includes a bone mineral density measurement area (the second lumbar vertebra to the fourth lumbar vertebra in this example) indicated by a measurement range. For example, the template 1306 is a rectangle in which a bone mineral density measurement area is circumscribed and may be set similarly to the template 1201 (FIG. 12) of the second embodiment. A search area 1305 for local matching is set in the second measurement area image 1302 based on the global shift amount obtained by global matching and the bone mineral density measurement area. For example, after moving the second measurement area image 1302 by the global shift amount, the search area 1305 is determined based on the bone mineral density measurement area (second to fourth lumbar vertebra). Since the alignment by global matching has been completed, the search area 1305 may be set to be smaller than the search area 1303. For example, a vertical size of the search area 1305 can be set similarly to the search area 1202 (FIG. 12) used in the second embodiment. In addition, since global matching has been completed, a horizontal size of the search area 1305 can be made smaller than the search area 1202 used in the second embodiment. By reducing a moving amount of the template 1306, calculation resources for local matching can be suppressed. The alignment unit 104 calculates a local shift amount (dxminL, dyminL) by local matching and then outputs this as a final shift amount (dxmin, dymin).

The bone image generating unit 105 generates a bone image 1310 by using the shift amount outputted from the alignment unit 104, the first measurement area image 1301, the second measurement area image 1302 (step S304). The bone mineral density calculating unit 106 calculates a bone mineral density of the bone mineral density measurement area based on the bone image 1310 (step S305). The processing of steps S304 and S305 are similar to those of the first and second embodiments.

As described above, in the third embodiment, by local matching being performed using a system-set measurement range (portion of the lumbar spine, which is a bone mineral density measurement area) after global matching in which alignment is performed on the entire lumbar spine, fast and accurate alignment can be performed. In addition, for example, by setting a template for global matching to be the entire lumbar spine and a template for local matching to be narrowed down to a lumbar spine mineral density measurement range, a fall into a local solution is prevented by global matching and the alignment accuracy is improved.

In the third embodiment, an example in which lumbar vertebrae are used as a bone mineral density measurement area has been described; however, the present invention is not limited to this, and for example, a head portion, a trochanter portion, or the like of a femur may be used as a bone mineral density measurement area. For example, in a case of a femur, global matching can be performed on a whole including bone portions including a pelvis portion, the head portion, and the trochanter portion, and local matching can be performed on the head portion and the trochanter portion.

### Fourth Embodiment

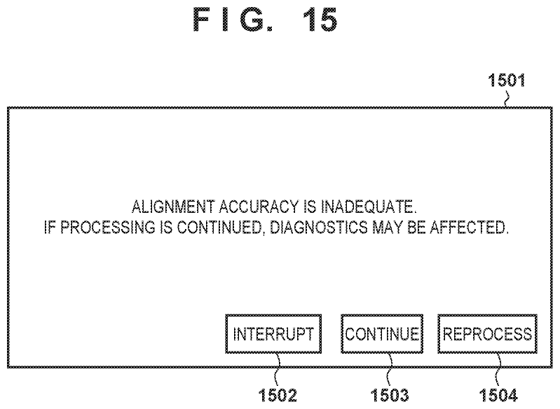

In the first embodiment to third embodiment, by narrowing down a partial area to be used as a template and a partial area to be used as a search area, the accuracy of alignment related to a bone mineral density measurement area is improved, and thereby the accuracy of bone mineral density calculation processing is improved. However, there are cases where sufficient alignment accuracy cannot be obtained depending on the image in a partial area to be a template or a search area. In a fourth embodiment, in such a case, alignment accuracy is improved by expanding a partial area for alignment processing. A functional configuration and a hardware configuration of a bone mineral density measurement apparatus of the fourth embodiment are similar to those of the first embodiment (FIGS. 1 and 2). FIG. 14 is a flowchart for explaining bone mineral density calculation processing according to the fourth embodiment. Step S1401 to step S1402 are similar to step S301 to step S302 of the first embodiment (FIG. 3). The alignment unit 104 performs alignment processing by using the first measurement area image and the second measurement area image (step S1403). In the processing of the first embodiment (step S303), simply, a shift amount (dxmin, dymin) for which an SSD value is the smallest is outputted. In contrast, the alignment unit 104 of the fourth embodiment evaluates alignment accuracy by using an SSD value and then determines whether the alignment accuracy is greater than or equal to a predetermined level (step S1404). The closer the SSD value is to 0, the higher the similarity; therefore, an SSD value (minimum SSD value) that is closest to 0 among SSD values obtained in step S1403 is compared with a threshold in order to evaluate alignment accuracy. The threshold used here may be a value set in advance in the system or may be a value arbitrarily set by the user. If the smallest SSD value is less than the threshold, it is determined that the alignment accuracy is greater than or equal to the predetermined level (YES in step S1404). In such a case, the bone image generating unit 105 generates a bone image by using a result of the alignment in step S1403 (step S1405), and the bone mineral density calculating unit 106 calculates a bone mineral density (step S1406). The processing of steps S1405 and S1406 are similar to that of steps S304 and S305 of the first embodiment (FIG. 3).

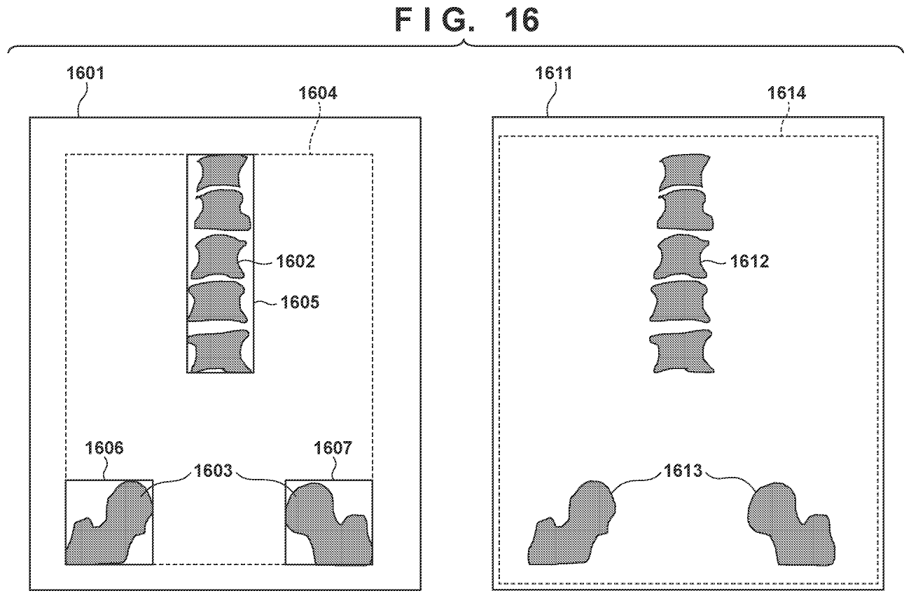

Meanwhile, when the smallest SSD value is greater than or equal to the threshold, it is determined that the alignment accuracy has not reached the predetermined level (NO in step S1404), and the alignment unit 104 asks the user whether to perform reprocessing for alignment (step S1407). For example, the alignment unit 104 displays a pop-up warning message 1501 as illustrated in FIG. 15 on the display unit 214 (FIG. 2). The warning message 1501 may indicate, for example, that alignment accuracy is low and if processing is continued, bone mineral density calculation accuracy may be low (“Alignment accuracy is inadequate. If processing is continued, diagnostics may be affected.”). The warning message 1501 also includes buttons 1502 to 1504 for selecting whether to continue, interrupt, or reprocess. When continuation (button 1503) is designated, the processing proceeds from step S1408 to step S1405, and a bone mineral density is calculated using the current alignment result (alignment result at the time of designation) (steps S1405 and S1406). In addition, when interruption (button 1502) is designated, the processing for bone mineral density calculation is not performed (steps S1405 and S1406 are skipped), and the processing ends.

If reprocessing (button 1504) is designated, a bone mineral density measurement area is updated by adding another bone portion area to the bone mineral density measurement area, and alignment is performed again based on the updated bone mineral density measurement area. First, the alignment unit 104 newly adds another bone portion as an alignment target (step S1409). For example, a bone portion, such as the sacrum, the pelvis, or the ribs, is added as an alignment target. Then, the alignment unit 104 updates the bone mineral density measurement area by adding a bone area of the alignment target bone portion to the current bone mineral density measurement area (step S1402). As a method of updating a bone mineral density measurement area, it is possible to use machine learning similar to that of the first embodiment, for example. At that time, for example, as described in the second embodiment, a learning model trained by using correct images in which an area in which another bone portion (the sacrum, the pelvis, the ribs, or the like) has been added to the lumbar spine has been manually labeled are used. As will be described later with reference to the fifth embodiment (FIG. 16), a configuration may be taken so as to individually set circumscribing rectangles and to use images within the set circumscribed rectangles as a template after the bone portion has been added. In addition, a search area is expanded by review and expansion according to addition of a bone portion. In addition, when re-executing the alignment processing, processing in which a bone portion is not added and only a search area is reviewed or expanded and then alignment is performed may be included. A configuration may be taken so as to, in step S1409, sequentially add bone portions, for example to add the sacrum in the first reprocessing, add the pelvis in the second reprocessing, and add the ribs in the third reprocessing, and update the bone mineral density measurement area. In addition, the bone portions added in the reprocessing are not subject to bone mineral density measurement, and so, in step S1406, a bone mineral density is calculated for the bone mineral density measurement area first set in step S1402 (i.e., the bone mineral density measurement area prior to being updated).

As described above, according to the fourth embodiment, when sufficient accuracy is not obtained by alignment in which a template and a search area obtained from a bone mineral density measurement area are used, a partial area for alignment processing is expanded and then alignment is repeated, and so, alignment accuracy is improved. In the fourth embodiment, a case where a bone mineral density measurement area is the lumbar spine is described; however, the prevent invention is not limited thereto, and another bone portion (e.g., the femora) may be set as the bone mineral density measurement area.

### Fifth Embodiment

In the first embodiment to the fourth embodiment, one collective body part, such as the lumbar spine or the femora, is used as a bone mineral density calculation area, and it is assumed that each is obtained by separate instances of X-ray imaging In a fifth embodiment, a configuration in which a plurality of body parts, such as the lumbar spine and the femora are simultaneously captured as bone mineral density measurement areas and respective bone mineral densities are calculated will be described. A functional configuration, a hardware configuration, and the overall flow of bone mineral density measurement of a bone mineral density measurement apparatus of the fifth embodiment are similar to those of the first embodiment (FIGS. 1, 2, and 3). Hereinafter, bone mineral density measurement processing according to the fifth embodiment will be described with reference to FIG. 3 again. In FIG. 16, measurement area images obtained from X-ray images in which the lumbar spine and the femora are simultaneously captured are illustrated.

The first obtaining unit 101 and the second obtaining unit 102 obtains a first X-ray image and a second X-ray image, respectively, which have been captured such that the lumbar spine and the femora of a subject fit in an X-ray irradiation range (step S301). Next, the area setting unit 103 sets bone mineral density measurement areas in the first X-ray image and the second X-ray image and then generates a first measurement area image 1601 and a second measurement area image 1611 (step S302, FIG. 16). In this example, bone areas of a lumbar spine 1602 and femora 1603 are set as the bone mineral density measurement areas. As a method of setting a bone mineral density measurement area, it is possible to use machine learning similar to that of the first embodiment, for example. In such a case, correct images to be used for machine learning are images in which both areas of the lumbar spine and the femora have been manually labeled.

The alignment unit 104 sets a template 1604 that includes a plurality of bone mineral density measurement areas (in the example of FIG. 16, three bone mineral density measurement areas, which correspond to the lumbar spine 1602 and the left and right femora 1603). In addition, the alignment unit 104 sets a search area 1614, which includes the lumbar spine 1602 and the femora 1603, in the second measurement area image 1611. The alignment unit 104 calculates a shift amount by template matching in which the template 1604 and the search area 1614 are used (step S303). However, SSD value calculation of template matching is performed only in a plurality of circumscribing rectangles 1605 to 1607, in which respective bone mineral density measurement areas are circumscribed, in the template 1604. The bone image generating unit 105 generates a bone image based on the first measurement area image 1601, the second measurement area image 1611, and the shift amount (step S304). Bone image generation is similar to that of the first embodiment. The bone mineral density calculating unit 106 calculates respective bone mineral densities of the bone mineral density measurement areas based on the bone image (step S305). A method of calculating a bone mineral density is similar to that of the first embodiment. However, when measuring a bone mineral density of the lumbar spine, the bone mineral density is calculated for a bone area of the lumbar spine (e.g., an area of the lumbar spine 1602), and when measuring a bone mineral density of femora, a bone mineral density is calculated for bone areas of the femora (e.g., areas of the femora 1603). In the above, in the alignment, alignment is performed according to a template that includes the lumbar spine 1602 and the femora 1603 and a search area; however, the present invention is not limited to this. For example, alignment and bone mineral density calculation may be performed for each bone mineral density measurement area (e.g., for each of the lumbar spine and the femora). For example, in the above example, when calculating a bone mineral density at the lumbar spine, alignment may be performed in the bone mineral density measurement area of the lumbar spine 1602, and when calculating a bone mineral density at the femora, alignment may be performed in the bone mineral density measurement areas of the femora 1603.

### Sixth Embodiment

In the first to fifth embodiments, an example in which shift processing (linear alignment) by translation, rotation, or the like is used as the alignment processing has been described. In a sixth embodiment, an example in which nonlinear alignment is used will be described. A functional configuration, a hardware configuration, and the overall flow of bone mineral density measurement of a bone mineral density measurement apparatus of the sixth embodiment are similar to those of the first embodiment (FIGS. 1, 2, and 3). Hereinafter, bone mineral density measurement processing according to the sixth embodiment will be described with reference to FIG. 3 again.

The processing of steps S301, S302, and S305 are similar to those of the first embodiment. The alignment unit 104 performs alignment processing by using the first measurement area image and the second measurement area image (step S303). In the sixth embodiment, the alignment unit 104 performs nonlinear alignment, such as warping, rather than linear alignment. Points in a bone mineral density measurement area (in the lumbar spine) in the first measurement area image and points in a bone mineral density measurement area in the second measurement area image are designated. Then, a nonlinear transformation parameter for deforming the entire image so as to bring the first measurement area image closer to the second measurement area image is obtained.

The bone image generating unit 105 converts the first measurement area image by taking as input the nonlinear transformation parameter outputted by the alignment unit 104 and then generates a bone image by taking a logarithmic difference between the first measurement area image and the second measurement area image (step S304). When taking the difference, a difference of a low energy image from a high energy image is taken.

As described above, according to the present disclosure, it is possible to improve the accuracy of alignment for an area necessary for bone mineral density measurement.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2022-171582, filed Oct. 26, 2022 which is hereby incorporated by reference herein in its entirety.

What is claimed is:

1. An information processing apparatus comprising:
a determination unit configured to determine, for each of a first radiation image corresponding to a first radiation energy and a second radiation image corresponding to a second radiation energy different from the first radiation energy, a first bone area indicating a predetermined bone portion;
an alignment unit configured to perform an alignment between a first partial area including the first bone area in the first radiation image and a second partial area including the first bone area in the second radiation image; and
an obtaining unit configured to obtain a bone mineral density of the first bone area based on a difference image including the first bone area, the difference image being obtained based on a result of the alignment.

2. The information processing apparatus according to claim 1, wherein the alignment unit performs the alignment by template matching in which the first partial area determined in the first radiation image is set as a template and the second partial area determined in the second radiation image is set as a search area.

3. The information processing apparatus according to claim 1, wherein the result of the alignment is a moving amount for matching the first partial area in the first radiation image and an area corresponding to the first partial area in the second radiation image.

4. The information processing apparatus according to claim 1, wherein the first partial area is a rectangle in which the first bone area determined in the first radiation image is circumscribed, and the second partial area is an area that includes the first bone area determined in the first radiation image and is larger than the rectangle.

5. The information processing apparatus according to claim 1, wherein in each of the first radiation image and the second radiation image, the determination unit determines a second bone area including the first bone area and determines the first bone area based on an image of the second bone area.

6. The information processing apparatus according to claim 5, wherein the determination unit determines the second bone area by using a learning model obtained by machine learning and determines the first bone area based on a profile obtained by adding pixel values of the second bone area in a predetermined direction.

7. The information processing apparatus according to claim 5, further comprising:
a designation unit configured to accept a designation of a measurement range of a bone mineral density by a user,
wherein the determination unit determines, as the first bone area, an area corresponding to the measurement range in the second bone area.

8. The information processing apparatus according to claim 1,
wherein the determination unit further determines, in the first radiation image and the second radiation image, a second bone area that includes the first bone area and that is larger than the first bone area, and
wherein after aligning a partial area set in the first radiation image and a partial area set in the second radiation image based on the second bone area, the alignment unit aligns the first partial area and the second partial area set based on the first bone area.

9. The information processing apparatus according to claim 8, wherein the first partial area set based on the second bone area is larger than the first partial area set based on the first bone area and the second partial area set based on the second bone area is larger than the second partial area set based on the first bone area.

10. The information processing apparatus according to claim 1, further comprising:
an evaluation unit configured to evaluate an accuracy of the result of the alignment,
wherein in a case where the evaluation by the evaluation unit has not reached a predetermined level, the alignment unit updates the first bone area by adding another bone portion to the first bone area and performs the alignment again by using the updated first bone area.

11. The information processing apparatus according to claim 10, wherein the obtaining unit obtains the bone mineral density of the first bone area prior to the update.

12. The information processing apparatus according to claim 10, further comprising: a designation unit configured to, in a case where the evaluation by the evaluation unit does not reach the predetermined level, accept a designation of a user as to whether to perform the alignment again.

13. The information processing apparatus according to claim 12, in a case where the designation of the user is not a designation for performing the alignment and is a designation for continuing bone mineral density measurement, the obtaining unit obtains the bone mineral density of the first bone area based on a result of the alignment obtained at a point in time of the designation of the user.

14. The information processing apparatus according to claim 1, wherein the first bone area is a group of vertebral bodies selected from five vertebral bodies constituting a lumbar spine, and a size of the second partial area in a direction of an arrangement of the lumbar spine is a size, obtained by adding a size corresponding to one vertebral body to a rectangle in which the first bone area is circumscribed, or smaller.

15. The information processing apparatus according to claim 2,
wherein the first bone area includes a plurality of bone areas indicating a plurality of bone portions, for each of which a bone mineral density is obtained,
wherein in the template matching, an image of a plurality of rectangles set for the plurality of bone areas in the first partial area is used as the template.

16. The information processing apparatus according to claim 1, wherein the determination unit determines the first bone area in the first radiation image and the second radiation image by using a learning model obtained by machine learning.

17. A radiation imaging system comprising:
the information processing apparatus according to claim 1;
a radiation generation apparatus; and
a radiation imaging apparatus configured to capture the first radiation image and the second radiation image by using radiation irradiated from the radiation generation apparatus.

18. The radiation imaging system according to claim 17, wherein the radiation imaging apparatus captures the first radiation image while the radiation generation apparatus is generating radiation of the first radiation energy and captures the second radiation image while the radiation generation apparatus is generating radiation of the second radiation energy.

19. A method of controlling an information processing apparatus, the method comprising:
determining, for each of a first radiation image corresponding to a first radiation energy and a second radiation image corresponding to a second radiation energy different from the first radiation energy, a first bone area indicating a predetermined bone portion;
performing an alignment between a first partial area including the first bone area in the first radiation image and a second partial area including the first bone area in the second radiation image; and
obtaining a bone mineral density of the first bone area based on a difference image including the first bone area, the difference image being obtained based on a result of the alignment.

20. A non-transitory computer-readable storage medium storing a program for causing a computer to execute the method according to claim 19.
